(19) United States
(12) Patent Application Publication
(10) Pub. No.: US 2025/0114028 A1
(43) Pub. Date: Apr. 10, 2025

(54) OLFACTORY TEST DEVICE, OLFACTORY TEST SYSTEM, AND OLFACTORY TEST METHOD

(71) Applicant: SONY GROUP CORPORATION, TOKYO (JP)

(72) Inventors: YUKITO INOUE, TOKYO (JP); SHUJI FUJITA, TOKYO (JP); KAZUTAKA TAKAKI, TOKYO (JP); YUKI HASHIMOTO, TOKYO (JP)

(21) Appl. No.: 18/729,424

(22) PCT Filed: Oct. 19, 2022
(86) PCT No.: PCT/JP2022/038972

§ 371 Date: Jul. 16, 2024

(30) Foreign Application Priority Data
Jan. 25, 2022 (JP) 2022-009713

(51) Int. Cl. A61B 5/00; A61B 90/00; A61B 90/90

(52) U.S. Cl. CPC A61B 5/4011; A61B 90/08; A61B 90/90; A61B 2090/0803; A61B 2090/0808

(57) ABSTRACT

A device that is also applicable to an olfactory test is provided. The present technology provides an olfactory test device including: a main body; and a cartridge that holds an odor component, in which the main body includes a cartridge holding unit that holds the cartridge, a detection unit that detects an attachment status of the cartridge with respect to the cartridge holding unit, and an output unit that outputs a detection signal of cartridge attachment in the detection unit. The present technology also provides an olfactory test system including: the olfactory test device; and an information processing device including a processing unit that manages a state of the cartridge on the basis of the detection signal output from the output unit.

### TECHNICAL FIELD

The present technology relates to an olfactory test device, an olfactory test system, and an olfactory test method.

### BACKGROUND ART

In the related art, a technique has been proposed in which air is supplied to a storage device in which an odor component holding member that holds an odor component is stored, and a vaporized odor component is released by a flow of air.

For example, Patent Document 1 proposes a scent dispensing device including: a cylindrical shielding tube whose internal cavity serves as a fragrance transport airway and that has an opening on a part of a side surface; a fragrance container that stores a fragrance inside, is provided on an outer surface of the shielding tube, and has an opening on a side surface on the shielding tube side; and a blowing means capable of blowing air to one end of the fragrance transport airway, in which the interior of the fragrance container and the fragrance transport airway can be brought into a penetrating state by overlapping the opening of the shielding tube with the opening of the fragrance container through a relative rotational movement while the shielding tube and the fragrance container are in close contact with each other, and the interior of the fragrance container and the fragrance transport airway can be brought into a non-penetrating state by overlapping one opening of the shielding tube and the fragrance container with a portion other than the one opening through a relative rotational movement while the shielding tube and the fragrance container are in close contact with each other.

According to the scent dispensing device described in Patent Document 1, unintended release of fragrance can be suppressed, and scent can be dispensed at an appropriate timing.

### CITATION LIST

### Patent Document

Patent Document 1: Japanese Patent Application Laid-Open No. 2013-094436

### SUMMARY OF THE INVENTION

### Problems to be Solved by the Invention

In medical settings and the like, olfactory tests are carried out according to various diseases and ages. In recent years, anosmia has become a problem as a symptom of COVID-19, and the demand for olfactory tests in medical settings and the like is increasing.

Therefore, a main object of the present technology is to provide a device that can also be applied to an olfactory test.

### Solutions to Problems

First, the present technology provides an olfactory test device including:
a main body; and a cartridge that holds an odor component,
   in which the main body includes
   a cartridge holding unit that holds the cartridge,
   a detection unit that detects an attachment status of the cartridge with respect to the cartridge holding unit, and
   an output unit that outputs a detection signal of cartridge attachment in the detection unit.

The cartridge of the olfactory test device according to the present technology may include a first identification unit that identifies cartridge information.

The cartridge holding unit of the olfactory test device according to the present technology may include a second identification unit that identifies information regarding the cartridge holding unit.

The cartridge and/or the cartridge holding unit of the olfactory test device according to the present technology may include a deformation mechanism that deforms when the cartridge is attached.

Next, the present technology provides an olfactory test system including:
an olfactory test device including a main body, and a cartridge that holds an odor component,
the main body including
a cartridge holding unit that holds the cartridge,
a detection unit that detects an attachment status of the cartridge with respect to the cartridge holding unit, and
an output unit that outputs a detection signal of cartridge attachment in the detection unit; and
an information processing device including a processing unit that manages a state of the cartridge on the basis of the detection signal output from the output unit.

The cartridge of the olfactory test device may include a first identification unit that identifies cartridge information. In this case, the olfactory test system according to the present technology may further include a reading unit that reads the cartridge information from the first identification unit.

The processing unit of the information processing device may manage the state of the cartridge on the basis of the cartridge information acquired by the reading unit. In this case, the state of the cartridge may be one or more states selected from an attachment status, a number of times of use, and a period of use of the cartridge.

The olfactory test system according to the present technology may further include a display unit that displays an alert in a case where the state of the cartridge exceeds a specific threshold value.

The cartridge holding unit of the olfactory test device may include a second identification unit that identifies information regarding the cartridge holding unit. In this case, the reading unit may read the information regarding the cartridge holding unit from the second identification unit.

The information processing device of the olfactory test system according to the present technology may include a control unit that controls removal of the cartridge.

The cartridge and/or the cartridge holding unit of the olfactory test device may include a cartridge removal locking mechanism. In this case, the cartridge removal locking mechanism may set or release a cartridge removal lock on the basis of a control command from the control unit.

In the setting of the cartridge removal lock by the cartridge removal locking mechanism, the lock setting may be maintained even when power supply of the olfactory test device is turned off.

The control unit of the information processing device may control removal of the cartridge on the basis of reading information acquired by the reading unit.

Further, the present technology provides an olfactory test method including:
a detection step of detecting an attachment status of a cartridge holding an odor component with respect to a cartridge holding unit;
a reading step of reading information of the cartridge; and
a management step of managing a state of the cartridge on the basis of cartridge information acquired in the reading step.

### BRIEF DESCRIPTION OF DRAWINGS

FIG. 1 is a block diagram illustrating an embodiment of an olfactory test system 100 according to the present technology.

FIG. 2 is a block diagram illustrating an embodiment different from FIG. 1 of the olfactory test system 100 according to the present technology.

FIG. 3 is an exploded schematic view illustrating an embodiment of an olfactory test device 1 according to the present technology.

FIG. 4 is a conceptual view illustrating an operation when attaching a cartridge 10 to a cartridge holding unit 11.

FIG. 5 is a six-sided view illustrating an embodiment of the cartridge 10 of the olfactory test device 1 according to the present technology.

FIG. 6 is a cross-sectional view of the cartridge 10 along line A-A.

FIG. 7 is a cross-sectional view of the cartridge 10 along line B-B.

FIG. 8 is a conceptual cross-sectional view illustrating a state in which a glass tube 102 is crushed and an odor component held in the glass tube 102 is released from the glass tube 102.

FIG. 9 is a conceptual cross-sectional view illustrating a state in which an odor is released from the cartridge 10.

I of FIG. 10 is a schematic view illustrating an embodiment of a cartridge removal locking mechanism 16 of the olfactory test device 1 according to the present technology, and II of FIG. 10 is an end view viewed from above at a position of a convex portion 1011 of IC.

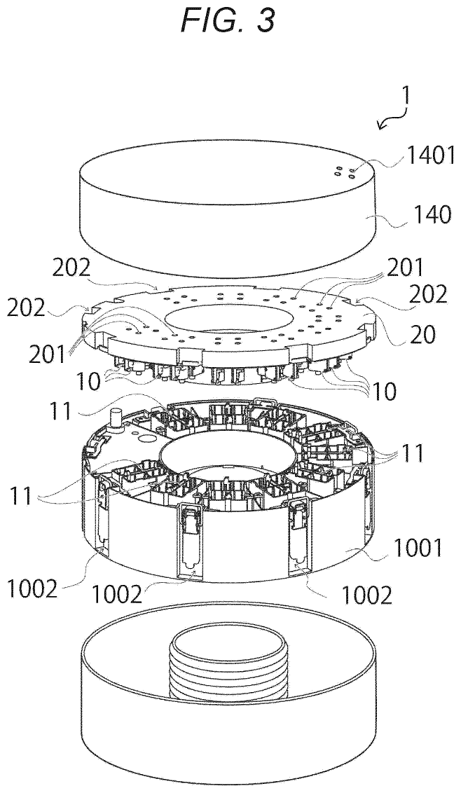

A of FIG. 11 is a schematic plan view of a cartridge unit 20 and a main body 1001 of the olfactory test device 1 according to the embodiment illustrated in FIG. 3 as viewed from a blower outlet 201 side, and B of FIG. 11 is a schematic plan view illustrating an example in which the convex portion 1011 is provided in the main body 1001 of A.

FIG. 12 is a plan view of the cartridge unit 20 and the main body 1001 of the olfactory test device 1 according to the embodiment illustrated in FIG. 3 as viewed from the blower outlet 201 side, and is a schematic view illustrating an arrangement of 10 locations when odor components are released.

FIG. 13 is a view in which the arrangements of the 10 locations in FIG. 12 are overlapped and portions of grooves 202 and 1012 are removed.

FIG. 14 is a flowchart illustrating an operation example of the olfactory test system 100 according to the present technology.

### MODE FOR CARRYING OUT THE INVENTION

Hereinafter, preferred modes for carrying out the present technology will be described with reference to the drawings. Embodiments to be described hereinafter illustrate examples of representative embodiments of the present technology, and any embodiments can be combined. Furthermore, the scope of the present technology should not be interpreted narrowly due to these. Note that the description will be made in the following order.
1. Olfactory Test System 100
   (1) Overall Configuration
   (2) Olfactory Test Device 1
      (2-1) Cartridge 10
         (2-1-1) First Identification Unit 110
      (2-2) Cartridge Holding Unit 11
      (2-3) Detection Unit 13
      (2-4) Output Unit 14
      (2-5) Second Identification Unit 111
      (2-6) Deformation Mechanism 15
      (2-7) Cartridge Removal Locking Mechanism 16
   (3) Information Processing Device 200
      (3-1) Processing Unit 2001
      (3-2) Control Unit 2002
   (4) Reading Unit 3
   (5) Display Unit 4
   (6) Storage Unit 5
   (7) User Interface 6
2. Operation Example of Olfactory Test System 100
3. Olfactory Test Method

### 1. Olfactory Test System 100

### (1) Overall Configuration

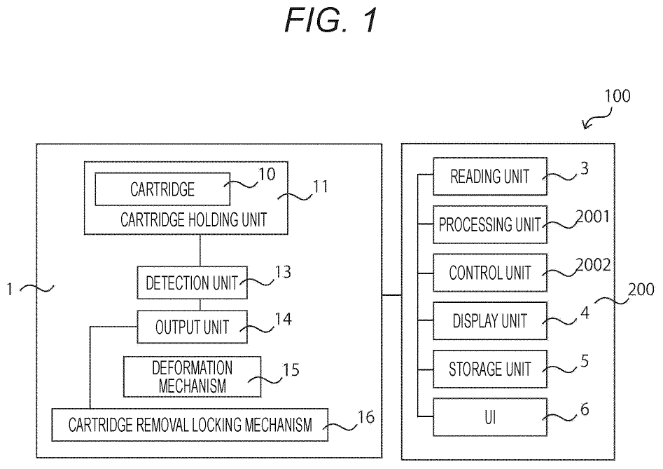

FIG. 1 is a block diagram illustrating an embodiment of an olfactory test system 100 according to the present technology. The olfactory test system 100 according to the present technology includes an olfactory test device 1 and an information processing device 200.

The olfactory test device 1 includes a cartridge 10, a cartridge holding unit 11, a detection unit 13, and an output unit 14. In addition, a deformation mechanism 15, a cartridge removal locking mechanism 16, and the like can be provided as necessary.

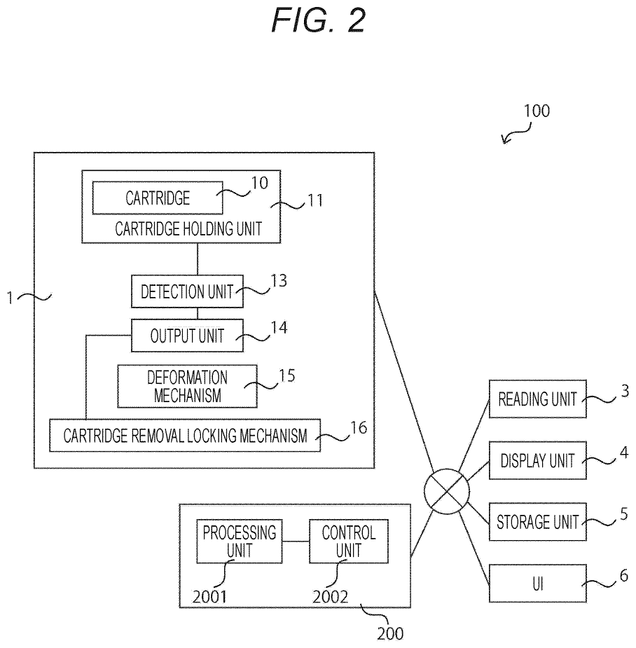

The information processing device 200 includes a processing unit 2001. Furthermore, a control unit 2002, a reading unit 3, a display unit 4, a storage unit 5, a user interface 6, and the like can be provided as necessary. Note that, as illustrated in FIG. 2, the reading unit 3, the display unit 4, the storage unit 5, the user interface 6, and the like may be provided outside the information processing device 200, and the olfactory test device 1, the information processing device 200, and the respective units may be connected via a network. In addition, although not illustrated, the reading unit 3, the display unit 4, and the user interface 6 may be provided in the olfactory test device 1, and the information processing device 200 and the storage unit 5 may be provided outside such as on a cloud and connected via a network. Furthermore, although not illustrated, the olfactory test device 1 can be provided with an information processing function. That is, the olfactory test device 1 can include the processing unit 2001, and the control unit 2002, the reading unit 3, the display unit 4, the storage unit 5, the user interface 6, and the like as necessary. Hereinafter, details of each device and each unit will be described.

### (2) Olfactory Test Device 1

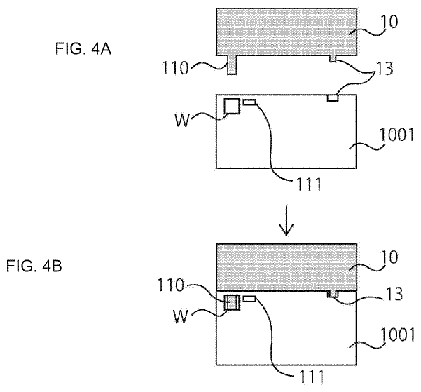

FIG. 3 is an exploded schematic view illustrating an embodiment of the olfactory test device 1 according to the present technology. FIG. 4 is a conceptual view illustrating an operation when attaching a cartridge 10 to a cartridge holding unit 11. The olfactory test device 1 according to the present technology includes a main body 1001 and a cartridge 10, and the main body 1001 includes at least a cartridge holding unit 11, a detection unit 13, and an output unit 14. In addition, a deformation mechanism 15, a cartridge removal locking mechanism 16, and the like can be provided as necessary.

### (2-1) Cartridge 10

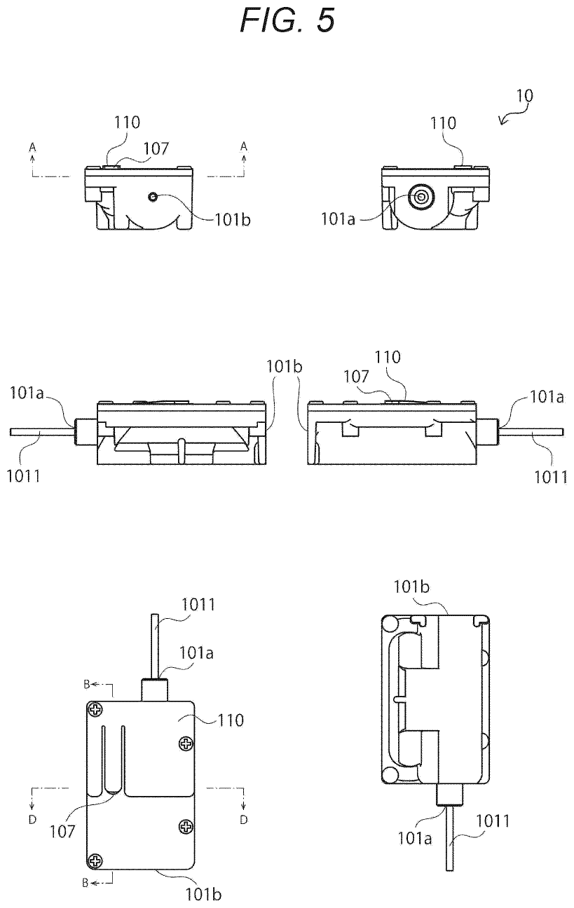

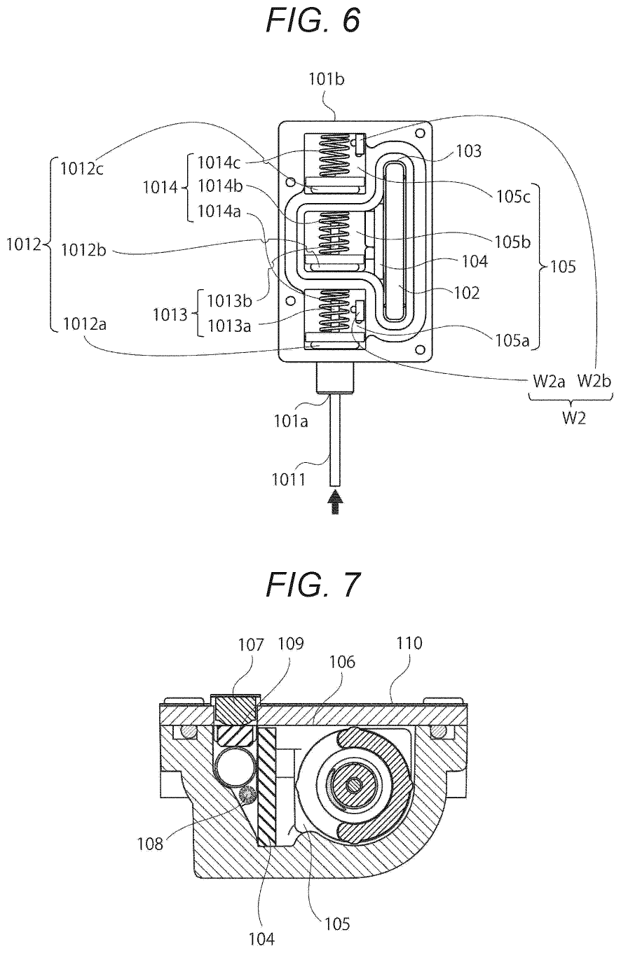

FIG. 5 is a six-sided view illustrating an embodiment of the cartridge 10 of the olfactory test device 1 according to the present technology (hereinafter also referred to as “cartridge 10”). FIG. 6 is a cross-sectional view of the cartridge 10 along line A-A. FIG. 7 is a cross-sectional view of the cartridge 10 along line B-B.

The cartridge 10 used in the present technology includes at least an opening 101 (101a, 101b), a glass tube 102, a glass tube holding portion 103, an odor holding body 104, and a ventilation portion 105. Furthermore, a pressing portion 107 and the like can be provided as necessary.

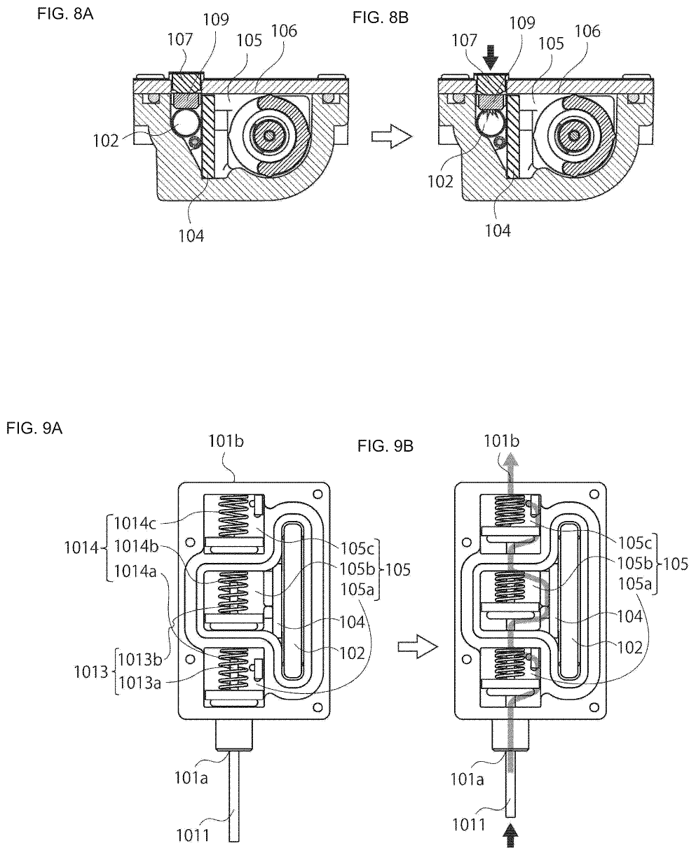

FIG. 8 is a conceptual cross-sectional view illustrating a state in which the glass tube 102 is crushed and an odor component held in the glass tube 102 is released from the glass tube 102. If the pressing portion 107 is pressed, pressure is applied to the glass tube 102, causing the glass tube 102 to be crushed. The odor component is released from the crushed glass tube 102, and the released odor component is absorbed and held by the odor holding body 104.

Note that the pressing portion 107 is not essential to the cartridge 10 used in the present technology, and for example, the glass tube 102 can be pressed by providing the olfactory test device 1 with a pressing function or using an external pressing device or the like.

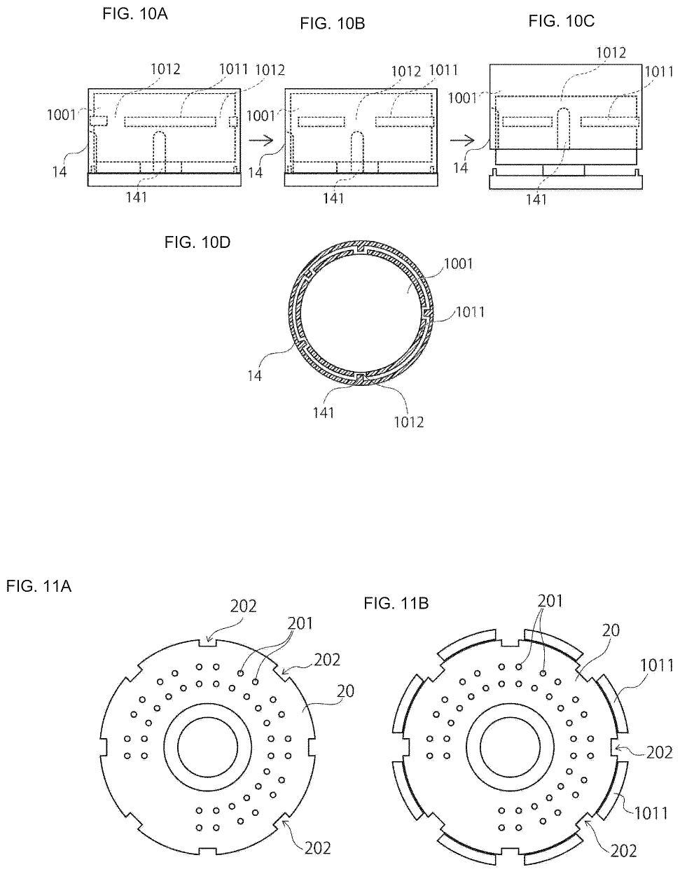

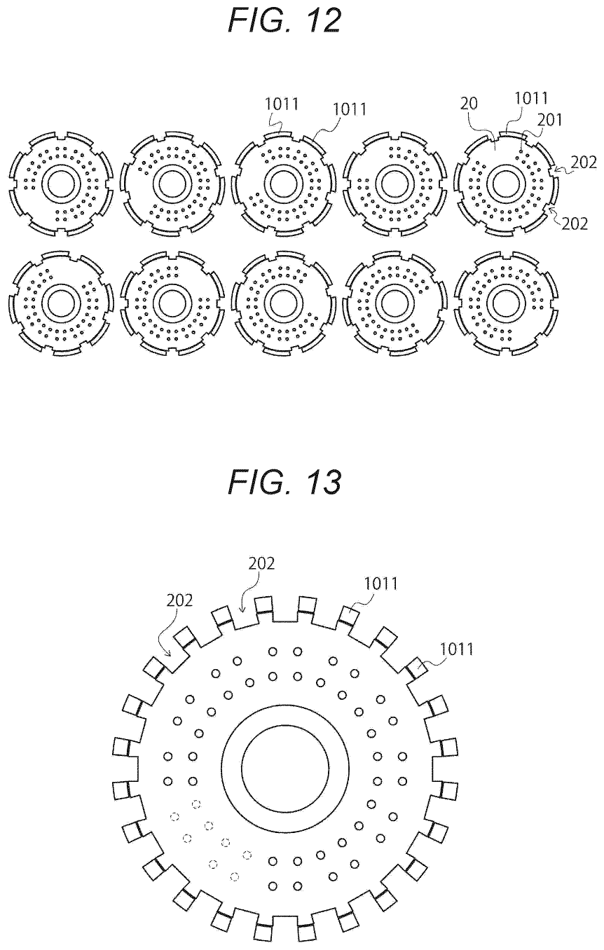

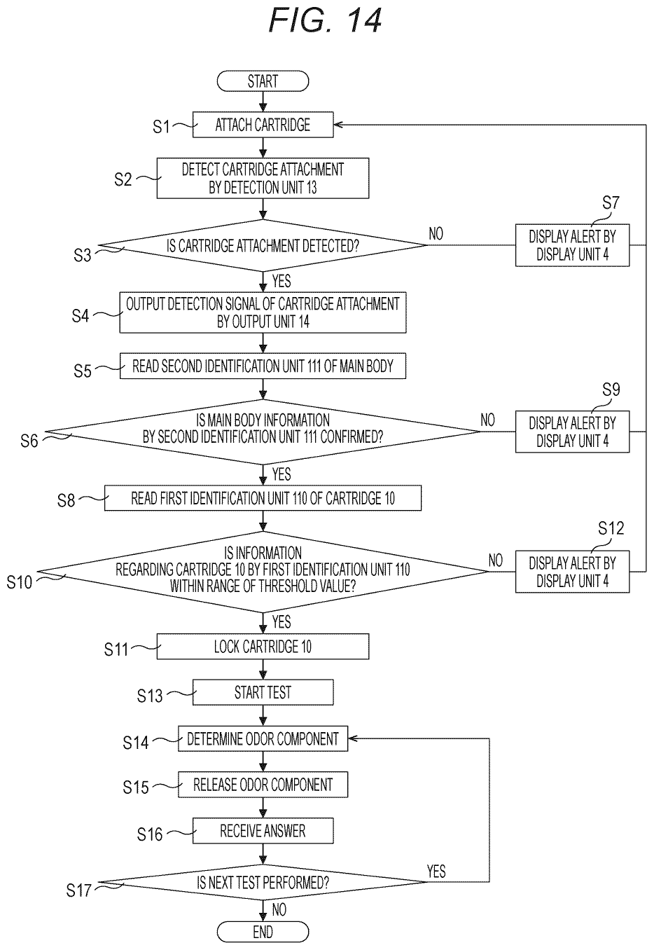

FIG. 9 is a conceptual cross-sectional view illustrating a state in which an odor is released from the cartridge 10 used in the present technology. When one opening 101a (inflow opening 101a) is opened in a state where the odor component is held by the odor holding body 104, the air flows in from the outside. The flowed air flows into the ventilation portion 105 and mixes with the odor component held by the odor holding body 104. In this state, by opening the other opening 101b (release opening 101b), the air containing the odor component is released to the outside.

The number of cartridges 10 used in the olfactory test device 1 according to the present technology is not particularly limited, and one or two or more cartridges 10 can be freely used according to the purpose of test or the like. For example, in a case where the olfactory test device 1 according to the present technology is used for the reference olfactory test in Japan, the number of cartridges 10 can be selected so that 40 (5 types of olfactory elements×8 concentrations) odors can be generated, and the number of odors conforming to the testing criteria and testing methods of that country can be generated outside Japan.

The cartridge 10 is used by being attached to the cartridge holding unit 11, which will be described later. In a case where the number of cartridges 10 is small, it is also possible to attach each of the cartridges to a predetermined cartridge holding unit 11 one by one, but in a case where the number of cartridges 10 to be attached is large, as illustrated in FIG. 3, it is also possible to attach a plurality of cartridges 10 to the olfactory test device 1 at a time by using a cartridge unit 20 in which a plurality of cartridges 10 is supported in advance.

### (2-1-1) First Identification Unit 110

The cartridge 10 can include a first identification unit 110 that identifies cartridge information. Examples of the cartridge information include a number for individually identifying the cartridge 10, a manufacturing number, a type of odor component held in the cartridge 10, a concentration of the odor component, a manufacturing date of the cartridge 10, and the like.

A specific configuration of the first identification unit 110 is not particularly limited as long as it can identify the cartridge information. Examples thereof include an IC chip, an IC tag, a barcode, a matrix-type two-dimensional barcode, and the like.

The reading unit 3, which will be described later, reads the cartridge information from the first identification unit 110, and the reading information is output to the processing unit 2001 in the information processing device 200, which will be described later.

### (2-2) Cartridge Holding Unit 11

The olfactory test device 1 according to the present technology includes a cartridge holding unit 11 that holds the cartridge 10. The cartridge holding unit 11 can hold one or two or more cartridges 10. The number of the cartridges 10 to be held is not limited, and can be freely set according to the method of using the olfactory test device 1.

For example, in a case where the olfactory test device 1 according to the present technology is used for the reference olfactory test in Japan, the structure of the cartridge holding unit 11 can be designed so that 40 (5 types of olfactory elements×8 concentrations) odors can be generated, and the number of odors conforming to the testing criteria and testing methods of that country can be generated outside Japan.

The form of the cartridge holding unit 11 is also not particularly limited, and can be freely designed according to the form of the cartridge 10 to be held. For example, it can be formed in a substantially rectangular parallelepiped shape, a substantially cylindrical shape, a substantially cubic shape, or the like.

Preferably, the cartridge holding unit 11 is provided with means for breaking the glass tube 102 in the cartridge 10. For example, in a case where the above-described cartridge 10 is not provided with the pressing portion 107, it is preferable that the cartridge holding unit 11 be provided with a pressing mechanism for pressing a portion of a sealed crushing portion 106 of the cartridge 10 in contact with the glass tube 102. Furthermore, for example, in a case where the above-described cartridge 10 is provided with the pressing portion 107, a mechanism for pushing the pressing portion 107 may be provided in the cartridge holding unit 11. Furthermore, in a case where the cartridge 10 is attached to the cartridge holding unit 11 while being slid, the pressing portion 107 of the cartridge 10 can be designed to be pushed in by the inner wall surface of the cartridge holding unit 11.

A material forming the cartridge holding unit 11 is not particularly limited, and an organic polymer material, for example, may form the cartridge holding unit 11. As the organic polymer material, for example, one or more materials of polyvinyl chloride, polyethylene, a phenol resin, an olefin resin, nylon, polyester, a synthetic rubber, a silicone resin, a natural rubber, a protein, a nucleic acid, a lipid, a polysaccharide, and the like may be used. In addition, for example, one or more types of materials of polymer resins such as an acrylic resin, a urethane resin, an ABS resin, a polyether ether ketone (PEEK) resin, a polyacetal (POM) resin, a fluororesin, a cycloolefin polymer resin, or a polyimide resin, metals such as stainless steel or aluminum, inorganic crystals such as quartz, glass, and the like may be used. Furthermore, the cartridge holding unit 11 may be formed to be porous, and as the porous material, for example, a mesh structure, cork, mesoporous silica, calcium carbonate, or the like can be used. Furthermore, a fiber structure, a layered structure (for example, clay minerals and the like), ceramic, and the like can also be used other than the porous material.

### (2-3) Detection Unit 13

The olfactory test device 1 according to the present technology includes a detection unit 13 that detects the attachment status of the cartridge 10 with respect to the cartridge holding unit 11. The configuration of the detection unit 13 is not particularly limited as long as it can detect the attachment status of the cartridge 10 with respect to the cartridge holding unit 11. For example, detection can be performed using a physical method of detecting by providing a portion that physically changes if the cartridge 10 is attached to the cartridge holding unit 11, an electronic method of electronically detecting that the cartridge 10 is attached to the cartridge holding unit 11, or the like.

### (2-4) Output Unit 14

The olfactory test device 1 according to the present technology includes an output unit 14 that outputs a detection signal of the attachment of the cartridge 10 in the detection unit 13. The detection signal of the attachment of the cartridge 10 is output to the processing unit 2001 of the information processing device 200, which will be described later.

### (2-5) Second Identification Unit 111

The main body 1001 of the olfactory test device 1 according to the present technology can include a second identification unit 111 that identifies main body information. In the olfactory test device 1 according to the present technology, the second identification unit 111 is not essential, but by including the second identification unit 111, it is possible to prevent information of the cartridge 10 not attached to the main body 1001 from being read as described later. Details will be described later.

Examples of the main body information include a mark indicating the main body 1001, a number for individually identifying the main body 1001, a manufacturing number of the olfactory test device 1 (main body 1001), a type of odor component that can be tested with the olfactory test device 1 (main body 1001), a concentration of the odor component, a manufacturing date of the olfactory test device 1 (main body 1001), and the like.

A specific configuration of the second identification unit 111 is not particularly limited as long as it can identify the main body information. Examples thereof include an IC chip, an IC tag, a barcode, a matrix-type two-dimensional barcode, and the like. In addition, as will be described later, the second identification unit 111 may be a simple marker or the like since it is only necessary to confirm at least that the portion is the main body 1001.

The reading unit 3, which will be described later, reads the main body information from the second identification unit 111, and the reading information is output to the processing unit 2001 in the information processing device 200, which will be described later.

### (2-6) Deformation Mechanism 15

The olfactory test device 1 according to the present technology can include a deformation mechanism 15 that deforms when the cartridge 10 is attached. In the olfactory test device 1 according to the present technology, the deformation mechanism 15 is not essential, but by including the deformation mechanism 15, it is possible to prevent the cartridge 10 after use from being erroneously reattached.

A specific configuration of the deformation mechanism 15 is not particularly limited as long as it can be deformed when the cartridge 10 is attached. For example, the deformation mechanism 15 may physically change if the cartridge 10 is attached to the cartridge holding unit 11, or the deformation mechanism 15 may electronically change such that electronic information is overwritten if the cartridge 10 is attached to the cartridge holding unit 11.

The deformation mechanism 15 may be provided in any of the cartridge 10, the cartridge unit 20, the cartridge holding unit 11, or other portions of the main body 1001, or may be provided at a plurality of locations.

### (2-7) Cartridge Removal Locking Mechanism 16

The olfactory test device 1 according to the present technology can include a cartridge removal locking mechanism 16. In the olfactory test device 1 according to the present technology, the cartridge removal locking mechanism 16 is not essential, but by including the cartridge removal locking mechanism 16, it is possible to prevent the cartridge 10 from being detached during the test.

In addition, in a case where the wrong cartridge 10 is attached, the cartridge removal locking mechanism 16 is designed not to be locked, and the test is designed not to be started in a state where the lock is released, so that it is possible to prevent unnecessary tests from being performed.

In the setting of the removal lock of the cartridge 10 by the cartridge removal locking mechanism 16, the lock setting is preferably maintained even when the power supply of the olfactory test device 1 is turned off. Accordingly, it is possible to prevent the cartridge 10 from being removed when the power supply is turned off, and it is also possible to prevent the cartridge 10 that has been erroneously removed from being attached again.

The cartridge removal locking mechanism 16 may be provided in any of the cartridge 10, the cartridge unit 20, the cartridge holding unit 11, or other portions of the main body 1001, or may be provided at a plurality of locations.

A specific configuration of the cartridge removal locking mechanism 16 is not particularly limited as long as it can lock removal of the cartridge 10. An example of the cartridge removal locking mechanism 16 will be described with reference to FIG. 10. For example, the removal of the cartridge 10 can be controlled by providing a rib 141 inside a front storage portion 140, providing a band-shaped convex portion 1011 having a plurality of spaces 1012 therebetween outside the main body 1001, and removing the front storage portion 140 in a case where the positions of the rib 141 and the space 1012 coincide with each other (see IB of FIG. 10).

More specifically, a configuration method of the cartridge removal locking mechanism 16 will be described using, for example, the case of the olfactory test device 1 according to the embodiment illustrated in FIG. 3 as an example. FIG. 11A is a plan view of the cartridge unit 20 and the main body 1001 of the olfactory test device 1 according to the embodiment illustrated in FIG. 3 as viewed from a blower outlet 201 side. In the olfactory test device 1 according to the present embodiment, since the cartridge unit 20 and the main body 1001 have grooves 202 and 1002 for providing buckles for fixing the cartridge unit 20 to the main body 1001 (see FIG. 3), the convex portion 1011 as illustrated in FIG. 10 cannot be provided in the portions of the grooves 202 and 1002 (see FIG. 11B).

In addition, in the olfactory test device 1 according to the present embodiment, a total of 40 cartridges 10 and cartridge holding units 11 are arranged, and the cartridge unit 20 and the main body 1001 are rotated so that the blower outlet 201 of the cartridge unit 20 matches a blower outlet 1401 of the front storage portion 140 at the time of releasing the odor component by setting four cartridges 10 and four cartridge holding units 11 each. In the olfactory test device 1 according to the present embodiment, since the four blower outlets 201 and 1401 are arranged for the 40 cartridges, the olfactory test device 1 stops at 10 different locations and releases odor components. FIG. 12 illustrates 10 different arrangements.

FIG. 12 is a plan view of the cartridge unit 20 and the main body 1001 of the olfactory test device 1 according to the embodiment illustrated in FIG. 3 as viewed from the blower outlet 201 side, and is a schematic view illustrating 10 different arrangements when odor components are released. Here, in the 10 different arrangements illustrated in FIG. 12, since odor components are released, it is preferable to configure the cartridge removal locking mechanism 16 such that it cannot be released.

FIG. 13 is a view in which the 10 different arrangements in FIG. 12 are overlapped and a portion of the groove 202 is removed. In FIG. 13, the convex portion 1011 always exists in a portion other than the groove 202. Therefore, if the rib 141 (see FIG. 10) is provided in the inner portion of the front storage portion 140 corresponding to the convex portion 1011 illustrated in FIG. 13, the locked state can be maintained without releasing the cartridge removal locking mechanism 16 in the arrangement in which the odor component is released.

### (3) Information Processing Device 200

The olfactory test system 100 according to the present technology includes an information processing device 200. The information processing device 200 includes a processing unit 2001. Furthermore, a control unit 2002, a reading unit 3, a display unit 4, a storage unit 5, a user interface 6, and the like can be provided as necessary. Hereinafter, details of each unit will be described.

### (3-1) Processing Unit 2001

The information processing device 200 of the olfactory test system 100 according to the present technology includes a processing unit 2001. In the processing unit 2001, the state of the cartridge 10 is managed on the basis of the detection signal of the attachment of the cartridge 10 output from the output unit 14 of the olfactory test device 1. Specifically, the processing unit 2001 can manage the attachment status of the cartridge 10 on the basis of the detection signal.

If the attachment of the cartridge 10 is confirmed, the processing unit 2001 can issue a reading instruction to the reading unit 3, which will be described later. In this way, only when the attachment of the cartridge 10 can be confirmed, it is possible to prevent the information of the cartridge 10 that has not been attached from being read by instructing the reading unit 3, which will be described later, to read.

The processing unit 2001 can also manage the state of the cartridge 10 on the basis of cartridge information acquired by the reading unit 3, which will be described later. Specifically, the processing unit 2001 can manage a number of times of use and a period of use of the cartridge 10 on the basis of the cartridge information acquired by the reading unit 3, which will be described later. In addition, the processing unit 2001 can also manage a number for individually identifying the cartridge 10, a manufacturing number, a type of odor component held in the cartridge 10, a concentration of the odor component, a manufacturing date of the cartridge 10, and the like on the basis of the cartridge information acquired by the reading unit 3, which will be described later.

The processing unit 2001 can issue a command to the display unit 4, which will be described later, to display an alert in a case where the state of the cartridge exceeds a specific threshold value. Specifically, in a case where the number of times of use of the cartridge 10 exceeds a predetermined number of times, in a case where the period of use of the cartridge 10 exceeds a predetermined period, in a case where the type of odor component held in the cartridge 10 is different from the component for the purpose of testing, in a case where the concentration of the odor component held in the cartridge 10 is different from the concentration for the purpose of testing, or the like, a command can be issued to the display unit 4, which will be described later, to display an alert.

### (3-2) Control Unit 2002

The information processing device 200 of the olfactory test system 100 according to the present technology can include a control unit 2002 that controls removal of the cartridge 10. The control unit 2002 issues a command to the cartridge removal locking mechanism 16 of the olfactory test device 1 described above to set or release the cartridge removal locking mechanism 16.

In the olfactory test system 100 according to the present technology, the control unit 2002 is not essential, but by including the control unit 2002, it is possible to prevent the cartridge 10 from being detached during the test.

In addition, in a case where the wrong cartridge 10 is attached, the control unit 2002 maintains the release of the cartridge removal locking mechanism 16, and the test is designed not to be started in a state where the lock is released, so that it is possible to prevent unnecessary tests from being performed.

As described above, in the setting of the removal lock of the cartridge 10 by the cartridge removal locking mechanism 16, the lock setting is preferably maintained even when the power supply of the olfactory test device 1 is turned off. Therefore, after the cartridge removal locking mechanism 16 of the olfactory test device 1 is set, it is preferable to design the test so that the lock setting is maintained unless the cartridge removal locking mechanism 16 is released by the control unit 2002 after the power supply of the olfactory test device 1 is turned off and the power supply is turned on again.

The control unit 2002 preferably controls the removal of the cartridge 10 on the basis of reading information acquired by the reading unit 3, which will be described later. For example, it can be designed to set the removal lock of the cartridge 10 by the cartridge removal locking mechanism 16 only in a case where the reading information of the cartridge 10 is within a range of a specific threshold value, and to maintain the unlock in a case where it is outside the range of the specific threshold value. By designing such that the test cannot be started in the unlocked state, it is possible to prevent unnecessary tests from being performed.

### (4) Reading Unit 3

The olfactory test system 100 according to the present technology can include a reading unit 3 that reads the cartridge information from the first identification unit 110 and/or the main body information from the second identification unit 111. The reading unit 3 is not essential to the olfactory test system 100 according to the present technology, but by including the reading unit 3, it is possible to contribute to prevention of erroneous attachment of the cartridge 10, prevention of attachment of the cartridge 10 after use, prevention of unnecessary test, and the like.

The reading by the reading unit 3 may be either the cartridge information from the first identification unit 110 or the main body information from the second identification unit 111, but it is preferable to read both of these pieces of information. For example, as illustrated in FIG. 4, a window W for reading the first identification unit 110 and the second identification unit 111 are provided adjacent to each other on the main body 1001, and in a case where these cannot be read at the same time, it is possible to prevent erroneous reading of the information of the cartridge 10 not attached to the main body 1001 by designing to issue an alert.

The information read by the reading unit 3 is output to the processing unit 2001 and the control unit 2002 of the information processing device 200.

Note that the installation location of the reading unit 3 is not particularly limited, and the reading unit 3 may be installed on the olfactory test device 1 side, on the information processing device 200 side, or on both sides. For example, in a case where the olfactory test device 1 has a reader function or the information processing device 200 has a camera function, the camera function can be used. In addition, the cartridge information from the first identification unit 110 and/or the main body information from the second identification unit 111 can be read using an external reading device.

### (5) Display Unit 4

The olfactory test system 100 according to the present technology can include a display unit 4. The display unit 4 can display all matters related to the olfactory test. For example, the number of times of use, the period of use, the type of odor component, the concentration of the odor component, the progress of the test, the test result, and the like of the attached cartridge 10 can be displayed. Note that, in the olfactory test system 100 according to the present technology, the display unit 4 is not essential, and all matters related to the olfactory test can be displayed using an external display device.

The display unit 4 can display an alert in a case where the state of the cartridge 10 exceeds a specific threshold value. For example, in a case where the number of times of use of the cartridge 10 exceeds a predetermined number of times, in a case where the period of use of the cartridge 10 exceeds a predetermined period, in a case where the type of odor component held in the cartridge 10 is different from the component for the purpose of testing, in a case where the concentration of the odor component held in the cartridge 10 is different from the concentration for the purpose of testing, or the like, an alert can be displayed.

Note that the installation location of the display unit 4 is not particularly limited, and the display unit 4 may be installed on the olfactory test device 1 side, on the information processing device 200 side, or on both sides. For example, information for a subject can be displayed on the display unit 4 installed on the olfactory test device 1 side, and information for a laboratory technician, a nurse, a doctor, and the like can be displayed on the display unit 4 installed on the information processing device 200 side.

### (6) Storage Unit 5

The olfactory test system 100 according to the present technology can include a storage unit 5. The storage unit 5 can store all matters related to the olfactory test. For example, the number of times of use, the period of use, the type of odor component, the concentration of the odor component, the progress of the test, the test result, and the like of the attached cartridge 10 can be stored. Note that, in the olfactory test system 100 according to the present technology, the storage unit 5 is not essential, and all matters related to the olfactory test can be stored using an external storage device.

Note that the installation location of the storage unit 5 is not particularly limited, and the storage unit 5 may be installed on the olfactory test device 1 side, on the information processing device 200 side, or on both sides. Furthermore, it is also possible to install the storage unit 5 outside such as on a cloud and connect the storage unit 5 to the olfactory test device 1 or the information processing device 200 via a network.

### (7) User Interface 6

The olfactory test system 100 according to the present technology can include a user interface 6. The user interface 6 is a part to be operated by a user such as a subject, a laboratory technician, a nurse, or a doctor. The user can access each unit through the user interface 6 and control each unit of the olfactory test system 100 according to the present technology. Note that in the 100 according to the present technology, the user interface 6 is not essential, and an external operating device may be connected. As the user interface 6, for example, a mouse, one or more buttons, a keyboard, a touch panel, or the like can be used.

Note that the installation location of the user interface 6 is not particularly limited, and the user interface 6 may be installed on the olfactory test device 1 side, on the information processing device 200 side, or on both sides. For example, the user interface 6 installed on the olfactory test device 1 side can be set to perform an operation for a subject, and the user interface 6 installed on the information processing device 200 side can be set to perform an operation for a laboratory technician, a nurse, a doctor, or the like.

### 2. Operation Example of Olfactory Test System 100

FIG. 14 is a flowchart illustrating an operation example of the olfactory test system 100 according to the present technology. First, the cartridge 10 is attached (S1). If the attachment of the cartridge 10 is detected by the detection unit 13 (S2) and the attachment of the cartridge 10 is detected (S3), a detection signal of the cartridge attachment by the output unit 14 is output to the information processing device 200 (S4).

If the cartridge attachment is detected, the processing unit 2001 of the information processing device 200 issues a command to the reading unit 3 to read the second identification unit 111 of the main body, and the reading unit 3 reads the second identification unit 111 of the main body (S5). On the other hand, in a case where the cartridge attachment cannot be confirmed (S6), the processing unit 2001 instructs the display unit 4 to display an alert (S7), and returns to the attachment of the cartridge 10 (S1).

The reading unit 3 outputs the main body information read from the second identification unit 111 to the processing unit 2001 of the information processing device 200, the processing unit 2001 issues a command to the reading unit 3 to read the first identification unit 110 of the cartridge 10 if the main body information by the second identification unit 111 can be confirmed (S6), and the reading unit 3 reads the first identification unit 110 of the cartridge 10 (S8). On the other hand, in a case where the main body information cannot be confirmed, the processing unit 2001 instructs the display unit 4 to display an alert (S9), and returns to attachment of the cartridge 10 (S1).

The reading unit 3 outputs the information regarding the cartridge 10 read from the first identification unit 110 to the processing unit 2001 of the information processing device 200, and the processing unit 2001 determines whether or not the information regarding the cartridge 10 by the first identification unit 110 is within a range of a threshold value (S10). In a case where the information regarding the cartridge 10 is within the range of the threshold value, the control unit 2002 issues a lock setting command to the cartridge removal locking mechanism 16, the cartridge 10 is locked (S11). Then, the processing unit 2001 issues a command to the olfactory test device 1 to start the test. On the other hand, in a case where the information regarding the cartridge 10 is outside the range of the threshold value, the processing unit 2001 instructs the display unit 4 to display an alert (S12), and returns to the attachment of the cartridge 10 (S1).

If the test by the olfactory test device 1 is started (S13), an odor component is determined (S14). The determination of the odor component can be performed by the processing unit 2001 in the information processing device 200 on the basis of the information regarding the cartridge 10 and various types of information stored in the storage unit 5, or can be performed by a user such as a laboratory technician, a nurse, or a doctor. If the odor component is determined, the odor component is released (S15), and the processing unit 2001 receives an answer from the subject (S16). On the basis of the answer from the subject, the processing unit 2001 or the user such as a laboratory technician, a nurse, or a doctor determines whether or not to perform the next test (S17). In a case where the next test is performed, the process returns to the determination of the odor component (S14). In a case where the next test is not performed, the process ends.

### 3. Olfactory Test Method

An olfactory test method according to the present technology is a method of performing at least a detection step of detecting an attachment status of a cartridge holding an odor component with respect to a cartridge holding unit, a reading step of reading information of the cartridge, and a management step of managing a state of the cartridge on the basis of cartridge information acquired in the reading step. In addition, if necessary, it is also possible to perform a cartridge removal control step, a display step of displaying all matters related to the olfactory test, a storage step of storing all matters related to the olfactory test, and the like.

Note that each step of the olfactory test method according to the present technology is the same as the method performed by each unit of the olfactory test system 100 according to the present technology described above, and thus the description thereof will be omitted here.

Note that the present technology can have the following configurations.
(1)

An olfactory test device including:
a main body; and
a cartridge that holds an odor component,
in which the main body includes
a cartridge holding unit that holds the cartridge,
a detection unit that detects an attachment status of the cartridge with respect to the cartridge holding unit, and
an output unit that outputs a detection signal of cartridge attachment in the detection unit.
(2)

The olfactory test device according to (1), in which the cartridge includes a first identification unit that identifies cartridge information.
(3)

The olfactory test device according to (1) or (2), in which the main body includes a second identification unit that identifies main body information.
(4)

The olfactory test device according to any one of (1) to (3), in which the cartridge and/or the cartridge holding unit includes a deformation mechanism that deforms when the cartridge is attached.
(5)

An olfactory test system including:
an olfactory test device including
a main body, and
a cartridge that holds an odor component,
the main body including
a cartridge holding unit that holds the cartridge,
a detection unit that detects an attachment status of the cartridge with respect to the cartridge holding unit, and
an output unit that outputs a detection signal of cartridge attachment in the detection unit; and
an information processing device including a processing unit that manages a state of the cartridge on the basis of the detection signal output from the output unit.
(6)

The olfactory test system according to (5),
in which the cartridge includes a first identification unit that identifies cartridge information, and
the olfactory test system further includes a reading unit that reads the cartridge information from the first identification unit.
(7)

The olfactory test system according to (6), in which the processing unit manages the state of the cartridge on the basis of the cartridge information acquired by the reading unit.
(8)

The olfactory test system according to (7), in which the state of the cartridge is one or more states selected from an attachment status, a number of times of use, and a period of use of the cartridge.
(9)

The olfactory test system according to (7) or (8), further including a display unit that displays an alert in a case where the state of the cartridge exceeds a specific threshold value.
(10)

The olfactory test system according to any one of (6) to (9),
in which the main body includes a second identification unit that identifies main body information, and
the reading unit reads the main body information from the second identification unit.
(11)

The olfactory test system according to any one of (5) to (10), in which the information processing device includes a control unit that controls removal of the cartridge.
(12)

The olfactory test system according to (11),
in which the cartridge and/or the cartridge holding unit includes a cartridge removal locking mechanism, and
the cartridge removal locking mechanism sets or releases a cartridge removal lock on the basis of a control command from the control unit.
(13)

The olfactory test system according to (12), in which in the setting of the cartridge removal lock by the cartridge removal locking mechanism, the lock setting is maintained even when power supply of the olfactory test device is turned off.
(14)

The olfactory test system according to any one of (11) to (13), in which the control unit controls removal of the cartridge on the basis of reading information acquired by the reading unit.
(15)

An olfactory test method including:
a detection step of detecting an attachment status of a cartridge holding an odor component with respect to a cartridge holding unit;
a reading step of reading information of the cartridge; and
a management step of managing a state of the cartridge on the basis of cartridge information acquired in the reading step.

### REFERENCE SIGNS LIST

100 Olfactory test system
1001 Main body
1 Olfactory test device
10 Cartridge
110 First identification unit
11 Cartridge holding unit
13 Detection unit
14 Output unit
111 Second identification unit
15 Deformation mechanism
16 Cartridge removal locking mechanism
20 Cartridge unit
201 Blower outlet of cartridge unit 20
202, 1002 Groove
1011 Convex portion
140 Front storage portion
1401 Blower outlet of front storage portion 140
200 Information processing device
2001 Processing unit
2002 Control unit
3 Reading unit
4 Display unit
5 Storage unit
6 User interface

1. An olfactory test device comprising:
a main body; and
a cartridge that holds an odor component,
wherein the main body includes
a cartridge holding unit that holds the cartridge,
a detection unit that detects an attachment status of the cartridge with respect to the cartridge holding unit, and
an output unit that outputs a detection signal of cartridge attachment in the detection unit.

2. The olfactory test device according to claim 1, wherein the cartridge includes a first identification unit that identifies cartridge information.

3. The olfactory test device according to claim 1, wherein the main body includes a second identification unit that identifies main body information.

4. The olfactory test device according to claim 1, wherein the cartridge and/or the cartridge holding unit includes a deformation mechanism that deforms when the cartridge is attached.

5. An olfactory test system comprising:
an olfactory test device including
a main body, and
a cartridge that holds an odor component,
the main body including
a cartridge holding unit that holds the cartridge,
a detection unit that detects an attachment status of the cartridge with respect to the cartridge holding unit, and
an output unit that outputs a detection signal of cartridge attachment in the detection unit; and
an information processing device including a processing unit that manages a state of the cartridge on a basis of the detection signal output from the output unit.

6. The olfactory test system according to claim 5,
wherein the cartridge includes a first identification unit that identifies cartridge information, and
the olfactory test system further comprises a reading unit that reads the cartridge information from the first identification unit.

7. The olfactory test system according to claim 6, wherein the processing unit manages the state of the cartridge on a basis of the cartridge information acquired by the reading unit.

8. The olfactory test system according to claim 7, wherein the state of the cartridge is one or more states selected from an attachment status, a number of times of use, and a period of use of the cartridge.

9. The olfactory test system according to claim 7, further comprising a display unit that displays an alert in a case where the state of the cartridge exceeds a specific threshold value.

10. The olfactory test system according to claim 6,
wherein the main body includes a second identification unit that identifies main body information, and
the reading unit reads the main body information from the second identification unit.

11. The olfactory test system according to claim 10, wherein the information processing device includes a control unit that controls removal of the cartridge.

12. The olfactory test system according to claim 11,
wherein the cartridge and/or the cartridge holding unit includes a cartridge removal locking mechanism, and
the cartridge removal locking mechanism sets or releases a cartridge removal lock on a basis of a control command from the control unit.

13. The olfactory test system according to claim 12, wherein in the setting of the cartridge removal lock by the cartridge removal locking mechanism, the lock setting is maintained even when power supply of the olfactory test device is turned off.

14. The olfactory test system according to claim 11, wherein the control unit controls removal of the cartridge on a basis of reading information acquired by the reading unit.

15. An olfactory test method comprising:
a detection step of detecting an attachment status of a cartridge holding an odor component with respect to a cartridge holding unit;
a reading step of reading information of the cartridge; and
a management step of managing a state of the cartridge on a basis of cartridge information acquired in the reading step.
